(19) United States
(12) Patent Application Publication
(10) Pub. No.: US 2023/0169888 A1
(43) Pub. Date: Jun. 1, 2023

(54) SYSTEM FOR SIMULATING THE BREATHING OF A LIVING BEING

(71) Applicant: Loewenstein Medical Technology S.A., Luxembourg (LU)

(72) Inventor: Petrus Johannes Josephus Borm, Eindhoven (NL)

(21) Appl. No.: 17/820,064

(22) Filed: Aug. 16, 2022

(30) Foreign Application Priority Data
Aug. 26, 2021 (DE) 102021004375.8

(51) Int. Cl. G09B 23/28

(52) U.S. Cl. CPC G09B 23/288

(57) ABSTRACT

A system for simulating the breathing of a living being, comprising at least a gas module and a control module. The control module is configured and designed, in a first simulation part, to mathematically simulate the breathing of a living being, and, in a second simulation part, to control the gas module on the basis of the mathematical simulation from the first simulation part.

### CROSS-REFERENCE TO RELATED APPLICATIONS

The present application claims priority under 35 U.S.C. § 119 of German Patent Application No. 102021004375.8, filed Aug. 26, 2021, the entire disclosure of which is expressly incorporated by reference herein.

### BACKGROUND OF THE INVENTION

### 1. Field of the Invention

The invention relates to a device and a method for simulating the breathing of a living being.

### 2. Discussion of Background Information

New functions of ventilators and also of other medical appliances are often based in the first instance on theoretical considerations of certain situations. In particular, the detection of certain situations, for example by a ventilator, is in most cases based on such considerations. In order to test such functions, there is a need for simulators that simulate breathing.

Furthermore, it may often be necessary to carry out quality checks on appliances, for example on diagnostic appliances too, in order to ascertain whether they correctly detect a situation. Here again, simulators are used. Finally, simulators are also important in the context of training. For example, it may be possible in particular to present and practice quite rare events.

The simulators known from the prior art often provide a rudimentary representation of the breathing of a living being, with the result that only basic respiratory situations can be presented. Moreover, these known simulators often rely principally on a mechatronic setup of the simulation, without establishing a more comprehensive basis behind the sequence of events.

In view of the foregoing it would be advantageous to have available a system which permits simple simulation of the breathing of a living being.

### SUMMARY OF THE INVENTION

The invention provides to a system for simulating the breathing of a living being, comprising at least a gas module and a control module. The control module is configured and designed, in a first simulation part, to mathematically simulate the breathing of a living being, and, in a second simulation part, to control the gas module on the basis of the mathematical simulation from the first simulation part.

In some embodiments, the system is characterized in that the control module comprises a simulation unit, which is configured and designed to mathematically simulate the breathing.

In some embodiments, the system is characterized in that the control module is configured and designed to control the gas module such that in the second simulation part the mathematical simulation of the first simulation part is converted into a physical simulation of the breathing of a living being.

In some embodiments, the system is characterized in that the gas module comprises at least one expiration unit and at least one inspiration unit, the expiration unit being configured to simulate an expiration of a living being, and the inspiration unit being configured to simulate an inspiration of a living being.

In some embodiments, the system is characterized in that the simulation unit is designed to calculate and/or simulate the pressure which is generated in the lungs by the simulated living being.

In some embodiments, the system is characterized in that the gas module is designed and configured to physically simulate the pressure which is generated in the lungs by the simulated living being.

In some embodiments, the system is characterized in that the gas module is connectable to a ventilator via a port.

In some embodiments, the system is characterized in that the expiration unit comprises at least one gas source and/or at least one fan. In some embodiments, the system is characterized in that the inspiration unit is configured and designed to generate an underpressure.

In some embodiments, the system is characterized in that the expiration unit comprises a plurality of gas sources, the expiration unit being configured and designed to provide a gas mixture.

In some embodiments, the system is characterized in that the expiration unit is configured and designed to make available, on the basis of the mathematical simulation, a gas mixture which corresponds to a gas composition of the exhaled air of a living being.

In some embodiments, the system is characterized in that a fan is arranged in the gas module, the fan serving both as expiration unit and as inspiration unit by a switching of valves and bypass lines arranged in the gas module.

In some embodiments, the system is characterized in that at least one pneumatic resistance is arranged in the gas module.

In some embodiments, the system is characterized in that the system comprises a sensor arrangement which is configured and designed to detect values of the respiration.

In some embodiments, the system is characterized in that the control module is configured and designed to incorporate the values detected via the sensor arrangement into the mathematical simulation. In some embodiments, the system is characterized in that the control module comprises an evaluation unit which is configured and designed to evaluate and/or analyze the values detected via the sensor arrangement.

In some embodiments, the system is characterized in that the evaluation unit is configured and designed to analyze the values detected via the sensor arrangement in order to ascertain whether the mathematical simulation is correctly implemented by the gas module.

In some embodiments, the system is characterized in that the inspiration unit and the expiration unit are designed as a combined unit.

In some embodiments, the system is characterized in that the system comprises an input unit via which data, values and/or information are input, wherein the data, values and/or information serve at least in part as specifications for the mathematical simulation.

In some embodiments, the system is characterized in that the input unit is configured and designed to input values and/or data and/or information from the evaluation unit into the simulation unit.

In some embodiments, the system is characterized in that the input unit is connected to at least one input module, the actual simulation being displayed via the input module.

In some embodiments, the system is characterized in that the system comprises a respiratory gas humidifier and/or a respiratory gas heater.

In some embodiments, the system is characterized in that the control module is configured and designed to at least partially control a ventilator on the basis of the mathematical simulation, wherein the ventilator is connected to a real person.

In some embodiments, the system is characterized in that the system is combinable with patient simulators.

In some embodiments, the system is characterized in that the simulation of the breathing also comprises the simulation of further physiological parameters.

In some embodiments, the system is characterized in that a mathematically simulated respiratory flow is physically simulated by at least one fan, and the simulated gas composition is achieved by at least one gas source.

The invention also provides a method for simulating the breathing of a living being, wherein in one method step the breathing of the living being is simulated in a first simulation part by a mathematical simulation and, in a further method step, in a second simulation part, a gas module is controlled on the basis of the mathematical simulation.

In some embodiments, the method is characterized in that the mathematical simulation is converted directly into commands, and the gas module is controlled on the basis of the commands.

In some embodiments, the method is characterized in that, in one method step, measurement values of respiration are captured via sensors and are incorporated into the mathematical simulation.

In some embodiments, the method is characterized in that the mathematical simulation is adapted and/or modified automatically on the basis of the captured measurement values.

In some embodiments, the method is characterized in that the measurement values relating to the breathing of a real person are used for the mathematical simulation.

It will be noted that the features individually presented in the claims can be combined with one another in any desired, technically meaningful way and show further refinements of the invention. The description additionally characterizes and specifies the invention in particular in conjunction with the figures.

It will also be noted that an “and/or” conjunction used herein between two features, and linking them to each other, is always to be interpreted as meaning that in a first embodiment of the subject matter according to the invention only the first feature may be present, in a second embodiment only the second feature may be present, and in a third embodiment both the first and the second feature may be present.

In the course of the invention, living beings are to be understood in particular as living beings who breath gas, for example air. In particular, these living beings are to be understood as mammals, in particular humans. In some embodiments, the invention relates explicitly to the simulation of the breathing in humans.

Ventilation is to be understood as breathing supported and/or specified by an external source. The external source can include, for example, mechanical ventilation, for example via a ventilator, and/or manual ventilation, for example by mouth-to-mouth ventilation or a breathing bag, and/or the gas supply via a compressed air cylinder.

A ventilator is to be understood as any appliance which supports the natural breathing of a user or patient, which takes over the ventilation of the user or living being (e.g. patient and/or neonate and/or premature baby) and/or which serves for respiration therapy and/or influences the respiration of the user or patient in some other way. This includes by way of example, but not exclusively, CPAP and BiLevel appliances, anesthesia appliances, respiration therapy appliances, ventilators (for use in hospitals, in non-hospital environments or in emergencies), high-flow therapy appliances and coughing machines. Ventilators can also be understood as diagnostic appliances for respiration. Diagnostic appliances can generally be used to detect medical and/or respiratory parameters of a living being. These also include appliances that are able to detect and optionally process medical parameters of patients in combination with respiration or only in relation to respiration.

Unless expressly described otherwise, a patient interface can be understood as any peripheral designed for interaction with a living being, in particular for therapeutic or diagnostic purposes. In particular, a patient interface can be designed as a mask of a ventilator or as a mask connected to the ventilator. This mask can be a full-face mask, i.e. enclosing the nose and mouth, or a nose mask, i.e. a mask enclosing only the nose. Tracheal tubes or cannulas and so-called nasal cannulas can also be used as mask or patient interface. In some cases, the patient interface can also be a simple mouthpiece, for example a tube, through which the living being/patient/user at least exhales and/or inhales.

The simulation of the breathing of the living being by the system is divided into two simulation parts. In a first simulation part, the patient's breathing is mathematically simulated, and, in a second simulation part, a gas module is controlled on the basis of the mathematical simulation.

In the second simulation part, provision can be made that the gas module converts the mathematical simulation into a physical simulation.

In addition, or alternatively, provision can be made that, in the second simulation part, the mathematical simulation is used to control a ventilator.

The mathematical simulation comprises at least a computer-aided calculation of the lungs and/or of the trachea or the breathing of the simulated living being, wherein at least the pressure situation and/or flow situation in the lungs and/or the trachea and/or the breathing of the living being is calculated and/or simulated. For example, provision is made that the breathing of the living being is simulated on the basis of specified values regarding the pressure and the flow of the gas. Alternatively or in addition, provision can also be made that weight, height, sex, body fat proportion, muscle proportion, age, diseases, lung volume, state of the alveoli (collapsed, hyperextended, opened, and to what extent), oxygen/CO2 exchange, tidal volume, respiratory rate are included in the simulation. Further specifications and/or sensor values and/or parameters, which can be included in the simulation, in particular in the mathematical simulation, are for example tidal volume, uptake of anesthetic gases, coughing episodes and/or efforts, muscle strain in relation to the lungs/breathing, compliance, change in compliance, as a function of time and/or pressure, activity/movements of muscle groups, fat groups, heart rate, changes of the heart rate, blood thinning, oxygen and/or CO2 uptake/release in the body, body temperature, hyperventilation and hypoventilation, elimination of anesthetic gases via the lungs or kidneys.

Provision can also be made that specifications concerning the course of the mathematical simulation can be input. For example, breathing problems or respiratory situations (respiratory events) such as apnea, alveolar collapse, gasping breathing, increased/reduced respiratory rate, situations comparable to snoring, etc. can be preprogrammed. These can then be mathematically simulated, for example after predefined times. A random generator can also be provided which incorporates respiratory situations randomly into the mathematical simulation. For example, it is possible to stipulate which respiratory situation or situations are to be simulated, optionally with further settings such as the extent of the situation, and the situations are randomly incorporated into the simulation via the random generator. Provision can also be made for the extent to be predefined via a random generator.

Alternatively or in addition, the mathematical simulation reacts to sensor values. For example, a continuous pressure (CPAP) is applied by a ventilator, whereupon the mathematical simulation incorporates this pressure into the calculation. For example, provision can be made that the mathematical simulation reacts only to sensor values, in particular to the pressure made available by a ventilator.

In the control of the gas module on the basis of the mathematical simulation, provision is for example made that the system reacts with a flow to the pressure of the ventilator.

In a simulation sequence, provision can also be made that a mathematical simulation is first of all effected on the basis of input specifications and is subsequently adapted by sensor values. Provision can also be made that the mathematical simulation incorporates the input stipulations and also sensor values. For example, provision can be made that the mathematical simulation reacts to the sensor values, for example as a reaction to a changed mode of operation of a connected ventilator.

Alternatively or in addition, the sensor values can also relate to the breathing of a real person or a real living being. For example, on the basis of the measured sensor values of the breathing of the real person, the real person can be tracked via the mathematical simulation and, optionally, supplementary values can also be simulated.

For example, sensor values relating to pressure, flow, frequency, volume and/or gas composition can be captured, and a mathematical simulation of the situation in the lungs is permitted on the basis of these values. The mathematical simulation can for example calculate additional values, data and/or information, such as the gas composition in the lungs and/or a proportion of collapsed/hyperextended/open alveoli and/or the lung volume and/or further parameters of the lungs.

In some embodiments, provision can be made that, besides breathing, other physical functions of the living being, for example body temperature, blood values and/or heart beat, can be at least mathematically simulated. If appropriate, such a mathematical simulation can be forwarded directly as signals to attached measuring devices, and/or provision can be made to attach further physical simulation modules, which convert the respective mathematical simulation into a physical simulation.

Provision is made that various aspects of the breathing of the living being can be simulated by the mathematical simulation. In particular, the pressure and flow of the breathing are able to be simulated. In some embodiments, provision is moreover made that gas composition, respiratory rate, respiratory situations, breathing problems, gas exchange, etc., can be simulated.

For the system, it is further provided that in the second simulation part a gas module is controlled on the basis of the mathematical simulation. The gas module can on the one hand be a device which effects a physical simulation of the breathing, comprising at least a gas pressure and gas flow.

Gas composition, respiratory rate, respiratory situations, breathing problems and/or gas exchange and/or further aspects of the breathing may be able to be physically simulated via the gas module.

Alternatively or in addition, the gas module can be designed as a ventilator, such that the mathematical simulation is used to at least influence the control of the ventilator.

Provision can be made that the ventilator is controlled on the basis of the mathematical simulation.

For example, at least the additional values/data/information, calculated via the mathematical simulation, are forwarded to the ventilator, such that the ventilation is optionally adapted on this basis.

If further physical functions besides breathing are also mathematically simulated, provision can be made that, in addition to the gas module, further physical simulators are arranged in the system and can convert the mathematical simulation into a physical simulation.

Alternatively or in addition, provision can be made for the mathematical simulation to be converted at least into electrical signals, which in turn can be detected and/or interpreted for example via sensors.

One aspect of the invention relates to a system which is configured and designed to simulate the breathing of a living being. The simulation of the breathing relates to the at least partial autonomous breathing and/or the at least partial predefined breathing of a living being.

Provision is made that at least the gas flow of the breathing is simulated in the form of pressure and/or flow through the system. This includes a flow of gas through a respiratory opening into the system, in order to simulate the inspiration by a living being, and a flow of gas through a respiratory opening out of the system, in order to simulate the expiration. In some embodiments, provision is made that the physiological gas exchange in the lungs is also simulated by the system. This at least entails simulation of oxygen being taken up from the gas and CO₂ being released into the gas.

The system is set up to predefine breathing on the basis of the mathematical simulation and/or to adapt the simulation on the basis of external sources, for example a connected ventilator.

The system according to the invention is configured and designed to be connected to any types of appliances that interact with a stream of gas.

These include, for example, any ventilators according to the prior art and/or diagnostic appliances for the analysis of breathing or of respiratory gas. Other gas sources, for example manual ventilation by mouth-to-mouth ventilation and/or a bag and compressed gas cylinders or compressed air, as is customary in respiratory protection and in the case of divers, can also be connected to the system.

In some embodiments, it is envisioned that breathing masks and/or patient interfaces are also connected to the system, optionally via an additional adapter (for example an artificial head). To be able to determine influences and/or properties of a mask, provision can be made that further sensors are arranged in the region of the mask, for example in the artificial head. Alternatively or in addition, provision can also be made that a mask is additionally simulated. The simulation of a mask takes place for example in the mathematical simulation, such that no additional modifications to the gas module are needed, for example in order to simulate a leakage. Alternatively or in addition, it is also possible to provide a controllable valve, by which respiratory gas is able to escape from the gas module without flowing in the direction of a connected ventilator, in order also to physically simulate a leakage.

Provision can also be made that a breathing mask and/or further patient interfaces are simulated via the mathematical simulation. As a result, the mathematical simulation acts as if the simulated living being, for example a human patient, is using a patient interface. A physical implementation of the mathematical simulation can also be accordingly reproduced via the gas module. For example, if a ventilator is connected to the gas module, the gas module can act on the ventilator as if a living being with a patient interface is connected to the ventilator. As regards the patient interface, parameters such as leakage, volume (for example mask volume) and/or pneumatic resistances can be included. Moreover, provision can be made that an additional filter, for example an HME filter, on the patient interface is included in the mathematical simulation. For example, the volume and the pneumatic resistance of the filter can be incorporated as parameters into the simulation. A further parameter can also be the O₂/CO₂ concentration or the general gas mixture in the mask, for example for the purpose of O₂/CO₂ washout or O₂/CO₂ accumulation in the mask. A further parameter can also be the dead space volume of the mask.

Provision can also be made that the system can be used for diagnosis of a patient. For example, the patient's breathing can be measured by the gas module via sensors, the control module being configured to simulate aspects of the patient's breathing via the mathematical simulation. For example, provision can be made that the mathematical simulation is used to simulate values/parameters/situations of the patient from the measured values that go beyond the pure measurement values. For example, a precise situation in the lungs of the patient can be mathematically simulated via the measurement values, wherein the mathematical simulation reproduces an at least approximate state.

The system is configured and designed to simulate the breathing of a living being, in particular of humans.

Provision is made that the gas flow which is routed out of the system, for example via a port via which the system can be connected to and/or interact with external appliances or means, resets the breathing of a living being.

In some embodiments, the pressure generated by the living being is simulated in particular. The mathematical simulation serves to calculate a pressure profile which corresponds to the lung contraction and lung expansion during breathing. This corresponds largely to the natural breathing of a human/mammal which, upon exhalation, generates a positive pressure in the lungs, by which the gas is forced out of the lungs, and, upon inhalation, sucks gas into the lungs by means of an underpressure. It will be noted here that the pressure generated by the lungs or the living being is so described. For example, if a ventilator which additionally generates a pressure is connected to the gas module, the pressure generated by the simulation is additionally subjected to the pressure of the ventilator. Thus, it may also happen that a positive pressure is measured constantly within the gas module. Through the simulation of the breathing, the pressure thus fluctuates around the pressure generated by the ventilator, depending on the compensation by the ventilator.

In some embodiments, the system is connected to input modules, for example a computer, laptop, tablet, mobile device (cell phone). These input modules may also serve, for example, to generate an augmented reality. For example, the values calculated by the mathematical simulation and/or corresponding items of information are then displayed on a tablet and, if appropriate, linked to regions of the body of a patient.

The invention also provides a method for simulating the breathing of a living being, in particular a human.

In one variant given as an example, the method comprises a method step involving the input of data for the mathematical simulation of breathing.

The input of data can be, for example, an input of specifications, for example pressure, flow, volume, rate, gas composition and/or respiratory situations and/or breathing problems to be simulated. Alternatively or in addition, the data can also be sensor data.

In one method step, the mathematical simulation of the breathing is provided on the basis of the input data.

The time profile of the breathing and the associated parameters are calculated on the basis of the inputs, for example with computer assistance. In some embodiments, the mathematical simulation comprises a calculation or simulation of additional breathing parameters and/or of the situation in the lungs and/or trachea of a real patient on the basis of detected sensor values.

In a subsequent method step, the mathematical simulation is translated into commands for controlling a gas module. These commands are transferred to a control unit, for example. The gas module is controlled on the basis of the commands. In some embodiments, provision is made that the commands are implemented such that the gas module converts the mathematical simulation into a physical simulation.

In some embodiments, provision is made that the commands derived from the mathematical simulation are transmitted to a ventilator, in order to adapt the operating mode of the ventilator.

A further method step comprises the capture, processing and/or evaluation of values relating to the physically simulated breathing and/or the breathing of a real living being. The values can then be evaluated and/or introduced into the mathematical simulation. For example, the values detected or captured by the sensors are evaluated with respect to the correct physical simulation.

Alternatively or in addition, the mathematical simulation is adapted, preferably automatically, on the basis of the detected values and/or the evaluation. In some embodiments, the values detected by the sensors also form the basis of the mathematical simulation, for example in order to simulate supplementary values, information items and/or data of the patient. In some embodiments, the values relating to breathing can also be supplemented by values, data and/or information relating to further functions of the body and/or properties of the living being.

In an exemplary embodiment of the method, at or before the start of the simulation, data, information and/or values are input which serve as specifications for the mathematical simulation of the breathing of a living being, preferably a human. On the basis of these specifications, the breathing of the living being is calculated or simulated in the mathematical simulation. The specifications concerning the mathematical simulation comprise, for example, at least values relating to pressure and flow of the breathing. For example, maximum and minimum pressures and flows can be defined, which are then included in the simulation of inspiration and expiration. Further specifications, for example relating to the respiratory rate, gas composition, gas temperature, gas humidity, tidal volume and/or gas exchange in the lungs, can be input in some embodiments.

Provision can also be made to define certain ranges for the specifications, for example in the form of a maximum value and minimum value and/or also in the form of a mean value and/or an ideal value.

The mathematical simulation can for example also comprise a random generator, which ensures a certain irregularity in the simulated breathing. For example, more realistic breathing can thus be simulated. For example, the random generator can randomly allow the values for pressure, flow, gas composition and/or tidal volume to fluctuate about a mean value.

Provision can also be made that specifications concerning respiratory events or respiratory situations can also be input and included in the mathematical simulation. For example, a number, duration and/or time can be defined for the occurrence of various respiratory situations. Specifications relating to the living being, for example weight, age, height, sex, previous diseases, etc., can also be provided as specifications.

In the course of the method, the mathematical simulation serves as a foundation for the control of a gas module. For example, the mathematical simulation is converted into commands, on the basis of which the gas module is controlled. For example, provision is made that, at the same time as the mathematical simulation, the current mathematical simulation state is converted directly into a command, such that over the course of time the gas module always represents the current mathematical simulation state. For example, provision is made that the gas module acts on a connected ventilator like a real living being, in particular in relation to breathing.

At the same time, measurement values are captured relating to the physically simulated breathing generated by the gas module, if appropriate in interaction with a connected ventilator. Provision can be made that these measurement values are evaluated directly. For example, the evaluation comprises an analysis of whether the breathing provided by the mathematical simulation is also correctly reproduced. By analysis of the measurement values, it is also possible to analyze whether and/or how a ventilator reacts to the simulated breathing. Provision can additionally be made that the result of the evaluation is included directly in the mathematical simulation. For example, provision can be made that reaction to an operating mode of the ventilator is effected via the mathematical simulation.

In some embodiments, provision can alternatively or additionally be made that measurement values of the breathing of a real person serve as specifications for the mathematical simulation. Provision can be made here that the measurement values of the real person are used as a basis of a mathematical simulation via which supplementary information, values and/or data of the breathing of the real person are simulated. Provision can be made that a ventilator (as gas module) is controlled for example on the basis of the supplementary information/data/values, and/or said supplementary information/data/values are forwarded to the ventilator, and the ventilator can optionally adapt the ventilation of the real person. For example, the situation in the lungs and/or trachea of the real person can be mathematically simulated on the basis of measurement values, wherein the supplementary information/values/data are not able to be detected by measurement or are able to be detected only with considerable effort.

In some embodiments, provision can be made that coughing is simulated via the mathematical simulation. Such a simulation can be converted into a physical simulation, for example using embodiments of the gas module with two fans.

Provision can also be made that various sizes and types of living being can be simulated. Depending on the size of the living being or on the lung volume and gas flow, suitable modifications are made to the gas module. For example, provision can be made that a large tidal volume is achieved by a multiplicity of fans or by particularly large and/or powerful fans.

In some embodiments, provision can be made that at least two fans are connected in series. At least two fans can have an opposite output direction. By means of such an arrangement, it is possible to afford a number of advanced simulations. With two fans working in opposite directions, it is possible to react quickly to rapidly changing ventilation pressures.

In the exemplary embodiments, which are discussed in the context of the figures, a ventilator for example is connected to the system. The connection is to be understood purely as an example and does not exclude a connection of the system to other appliances and/or means.

### BRIEF DESCRIPTION OF THE DRAWINGS

Further advantages and features of the present invention will become clear from the description of the illustrative embodiments, which are explained below with reference to the accompanying drawings. In the drawings,

FIG. 1 shows schematically an exemplary embodiment of the system of the invention;

FIG. 2 shows schematically a further exemplary embodiment of the system of the invention;

FIG. 3 shows schematically a third exemplary embodiment of the system of the invention;

FIG. 4 shows schematically an exemplary embodiment of the system of the invention in conjunction with a ventilator;

FIG. 5 shows schematically another exemplary embodiment of the system of the invention; and

FIG. 6 shows schematically another exemplary embodiment of the system of the invention in conjunction with a ventilator.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

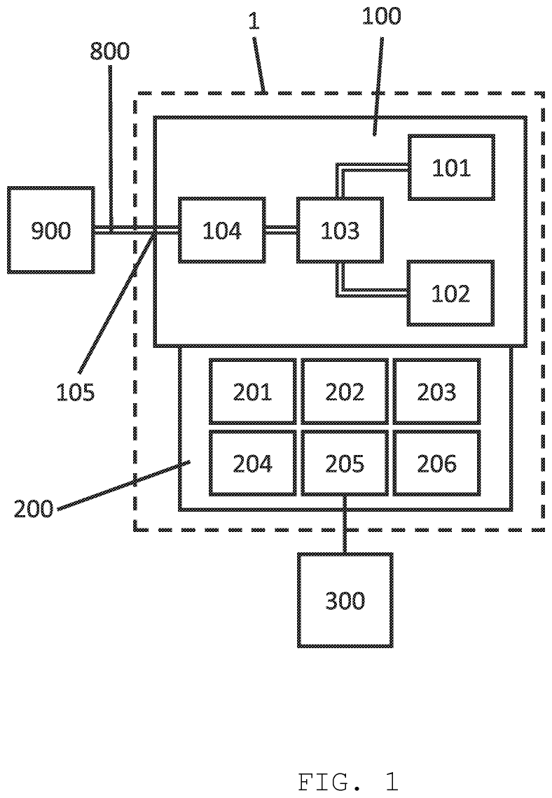

FIG. 1 shows schematically an exemplary embodiment of the system 1. The system 1 is configured and designed to simulate the breathing of a living being, for example a human. The simulation of the breathing comprises two simulation parts. In the first simulation part, the breathing of the living being is mathematically or computationally simulated in the control module 200. For the second simulation step, the gas module 100 is controlled by the control module 200 on the basis of the mathematical simulation. In the embodiment shown, provision is made for example that the mathematical simulation is converted into a physical simulation by the gas module 100.

For example, the system 1 is connected to a ventilator 900 via a connection. The ventilator 900 can be any type of ventilator according to the prior art. Besides using a ventilator, it is also possible here to apply ventilation quite generally, i.e. machine ventilation and/or manual ventilation (e.g. mouth-to-mouth, breathing bag). A ventilation using compressed air cylinders, as in respiratory protection or in the case of divers, can also be connected to the system 1. For example, in order to test the functionality of diagnostic appliances, it is also possible in some embodiments to connect corresponding diagnostic appliances to the system 1. By way of the connection 800, it is possible to establish a gas-conveying connection between the system 1 and the ventilator 900, for example via the port 105 of the gas module 100.

The gas module 100 of the system 1 comprises for example an expiration unit 101, an inspiration unit 102, an optional valve 103 for controlling or switching between inspiration and expiration, and also a sensor arrangement 104 for determining the respiratory parameters within the gas module 100. The expiration unit 101, designed for example as a compressed gas source and/or fan, is configured to simulate the expiration, i.e. to generate a gas stream which can escape from the system through the port 105 and corresponds, at least in terms of pressure profile and/or flow profile, to the breathing of a living being. For example, a pressure corresponding to the pressure generated in the lungs by a living being is generated via the expiration unit 101 and the inspiration unit 102.

The inspiration unit 102 is configured to simulate an inspiration of a living being. For this purpose, the inspiration unit 102 can for example comprise a fan and/or a vacuum pump and/or other devices with which, on the side of the port 105, an underpressure can be generated or gas is withdrawn from the system.

It will be noted at this point that, although fans can build up a pressure in one direction, the gas flow through a fan can in fact take place in two directions. To ensure that a gas flow takes place in the pressure direction via a fan, the fan has to overcome the counterpressure, generated for example by a ventilator, i.e. has to make available a higher pressure than the ventilator.

In some embodiments, the inspiration unit 102 is configured such that a valve is opened for example, optionally in conjunction with a pneumatic resistance and/or a variable volume, e.g. a balloon, by which a gas is able to flow through the port 105 into the system. By means of the pneumatic resistance or the volume, it is possible to simulate the filling of the lungs with gas by an external source, for example by ventilation/a ventilator 900. In some embodiments, the inspiration unit 102 can be switched such that an at least partially active inhalation and also a passive inhalation, i.e. defined by an external source, are simulated.

In some embodiments, provision is made that the expiration unit 101 and the inspiration unit 102 are formed as a combined unit which comprises both functions, i.e. simulation of inspiration and of expiration. In some embodiments, the inspiration unit 102 and/or the expiration unit 101 and/or the combined unit are designed to generate a defined gas mixture.

For example, provision is made that the expiration unit 101 not only simulates the gas flow but also simulates the capture of oxygen and release of CO2 in the lungs. For example, an optionally definable gas mixture is generated or mixed by the expiration unit 101.

Switching between simulated inspiration and expiration is realized, for example, by an optional valve 103. Optionally, the valve 103 is configured such that it is possible to switch steplessly between expiration unit 101 and inspiration unit 102. Alternatively, regulation via only the inspiration unit 102 and the expiration unit 101 is also possible, without an extra valve 103 having to be used.

In some forms of control, provision is made that a transition from inspiration to expiration, or vice versa, is simulated by the valve 103 switching in a suitably stepless manner. For example, a gradually decreasing gas flow at the end of expiration can be simulated, and, by varying the switching speed, the rapidly increasing gas flow of inspiration, compared to the decrease of the gas flow of expiration, can be adjusted. It should be noted here that the regulation can be impaired by frequent switching back and forth in the event of flows in the region of zero.

The sensor arrangement 104 is configured and designed to detect measurement values of the gas in the gas module 100. The measurement values relate for example to pressure, flow, temperature, humidity and/or gas composition. The sensor arrangement 104 is for example arranged between the expiration unit 101 or inspiration unit 102 and the port 105. The sensor arrangement 104 is arranged such that, by means of the detected measurement values, for example the gas parameters can be reproduced according to the lungs and/or the trachea of the simulated living being. By means of the sensor arrangement 104, it is possible for example to measure whether the breathing is simulated according to the specifications, for example the mathematical simulation. The influence of the ventilator 900, or of other devices and/or means connected via the port 105, on the breathing can also be detected.

For control of the gas module 100, the control module 200 comprises a control unit 201 which is designed to control at least the expiration unit 101 and the inspiration unit 102. If a valve 103 is provided for switching between expiration and inspiration, it is also controlled via the control unit 201. The control unit 201 is moreover configured and designed to control the gas module 100 on the basis of the mathematical simulation.

The control module 200 comprises a simulation unit 202 for the mathematical simulation of the breathing. The simulation unit 202 is configured and designed to mathematically simulate the breathing of a living being on the basis of specifications and/or inputs. In some embodiments, provision is made that the simulation unit 202, on the basis of the mathematical simulation of the breathing, generates control signals which are used by the control unit 201 to correspondingly control the gas module 100. For example, provision is made that the simulation unit 202 mathematically simulates the breathing in a first simulation part, wherein the mathematical simulation by the control unit 201 and the gas module 100 is converted into a physical simulation of the breathing.

The control module 200 of the system 1 moreover comprises for example a sensor unit 204, an evaluation unit 203, an input unit 205 and a storage unit 206. The sensor unit 204 is configured and designed to capture and optionally process the measurement values detected by the sensor arrangement 104. The evaluation unit 203 is configured and designed to evaluate and/or analyze the measurement values captured and optionally processed by the sensor unit 204. For example, provision can be made that the evaluation unit 203 analyzes the measurement values to ascertain whether the control of the gas module 100, as defined by the mathematical simulation, takes place correctly, for example whether the desired pressures, flows and/or volumes are generated. The results of the analysis and/or evaluation are for example forwarded via the input unit 205 to the simulation unit 202. By way of the simulation unit 202, the analysis results and/or also the measurement values themselves can be incorporated into the mathematical simulation, thus forming the basis of the control of the gas module 100.

For example, the control module 200 comprises a storage unit 206. Measurement values, analyses and/or evaluations can be stored at least on an intermediate basis in the storage unit 106. In some embodiments of the system 1, provision is made that the mathematical simulation and also the measurement values detected by the sensor arrangement 104 are stored in the storage unit 206, for example for a later comparison between mathematical simulation and physical simulation.

The input unit 105 serves for example s an interface via which data, values and/or information can be input into the system 1, in particular into the control module 200. In some embodiments, system-internal input of data, values and/or information also takes place via the input unit 205, for example from the evaluation unit 203, to the simulation unit 202. It is also envisioned that the input unit 205 is also configured and designed to forward data, values and/or information to an external appliance. For example, the input module 300 can be designed as a computer, notebook, smartphone and/or tablet and can be configured to display and optionally store values, data and/or information of the system 1.

The input module 300 is in particular configured to input into the system 1 specifications and/or settings relating to the simulation of the breathing. The simulation unit 202 is for example configured and designed to mathematically simulate the breathing of the living being on the basis of the specifications and/or settings. The specifications and/or settings comprise by way of example, and not exclusively, pressure, flow, lung volume, gas composition, respiratory rate, tidal volume, type of living being, age, weight, diseases (in particular respiratory diseases), gas exchange, breathing problems. In some embodiments, provision is made that the input module 300 has an input mask via which settings relating to the simulation of the breathing are input, which are transmitted to the control module 200. In some embodiments, a large number of simulation specifications and/or simulation sequences are stored in the storage unit 206 and can be accessed via the input module 300.

In some embodiments, provision is made that display of the actual simulation of the breathing, for example in the form of values and/or graphs, is possible via the input module 300.

In some embodiments, provision is made that a plurality of input modules 300 can be connected to the input unit 205 of the control module 200. For example, a connection to a plurality of sensors, actuators, a virtual reality and/or patient simulators is also possible. Moreover, provision is optionally made that input modules 300 can also serve for the output of values, data, information, displays, etc.

In some embodiments, the simulation of the breathing, in particular the mathematical simulation, also comprises the simulation of further physiological parameters. The simulation of further physiological parameters can comprise, for example, the blood circulation and/or the body temperature. Further physiological parameters or sequences can also optionally be included at least in the mathematical simulation and/or simulated. The simulation unit 202 is for example configured and designed such that the effect of the further physiological parameters on the simulation of the breathing can be incorporated into the mathematical simulation and corresponding control signals for the control unit 201 can be generated.

Moreover, provision can be made that, alternatively or in addition, an input module 300, for example comprising means of inputting and displaying data, values and information, is integrated into the system 100. Input means can be a keyboard and/or mouse for example. Display means can be a screen for example. It is also possible to provide a combined means of input and display, for example a touchscreen.

For the embodiment of the system 1 shown by way of example in FIG. 1, provision is made that the simulation of the breathing takes place entirely via the system 1. The simulation comprises the first simulation part (mathematical simulation) and the second simulation part (control of the gas module 100 for the physical simulation).

By way of the port 105 of the gas module 100, it is possible for example to connect a ventilator 900 to the system 1 or to the gas module 100. In comparison with the ventilator 900, the system 1 simulates for example the breathing of a living being.

The system 1 is configured and designed such that a large number of different respiratory situations or respiratory events can be simulated.

For example, in the simulation profile that is set, provision can be made that an airway obstruction is simulated. The ventilator 900 is for example configured and designed to react to an airway obstruction by increasing the ventilation pressure until the apnea is canceled. The sensor arrangement 104 detects this pressure increase by the ventilator 900 and forwards the measurement values to the control module 202. By way of the evaluation unit 204 and/or the simulation unit 202, the pressure increase effected by the ventilator 900 is analyzed and/or evaluated to ascertain whether the pressure increase is sufficient to treat or eliminate the airway obstruction. If the analysis and/or evaluation reveals that the pressure increase is sufficient to remove the airway obstruction, for example on the basis of a comparison with stored specifications, the simulation unit 202 accordingly adapts the mathematical simulation and the control signals for the physical simulation.

For the physical simulation of further aspects of the breathing or pulmonary function of a living being, the system 1 can additionally comprise a respiratory gas humidifier and/or a respiratory gas heater.

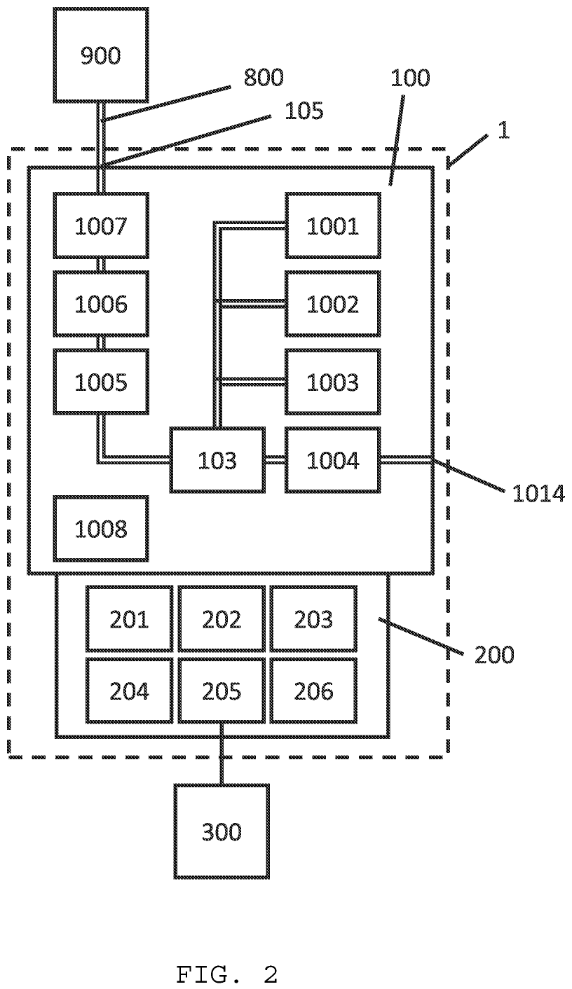

FIG. 2 shows schematically a further exemplary embodiment of the system 1. For the physical simulation of the expiration, the gas sources 1001, 1002, 1003 are provided, which together form the expiration unit 101 (see FIG. 1). For example, corresponding valves and pressure sensors (not shown explicitly) are arranged together with the gas sources 1001, 1002, 1003. For example, the valves assigned to the gas sources 1001, 1002, 1003 can be “open-close” valves, i.e. valves which are either open or closed. By means of a pulsed control, i.e. the opening or closing of the valves over a defined time period, a precise setting of the gas composition can be achieved. If the valves are opened per pulse for a defined time period, or for the pulse length, a defined volume flows through the valve. By fixing the pulse lengths and/or pulse numbers and/or pulse frequency, a precisely defined gas volume can flow through the valve, which can be utilized for a high degree of precision when setting the gas composition from the gas sources 1001, 1002, 1003. Alternatively or in addition, provision can be made that the valves of the gas sources 1001, 1002, 1003 are proportional valves.

By way of the gas sources 1001, 1002, 1003, a gas mixture can be made available for the physical simulation of the expiration, which gas mixture corresponds to the gas mixture that is exhaled by the simulated living being. For example, the uptake of oxygen from the respiratory air and the CO2 release into the respiratory air in the lungs can be simulated. For example, the gas source 1001 is designed as CO2 source, the gas source 1002 as oxygen source, and the gas source 1003 as nitrogen source. The gas mixture can be specifically adjusted, for example, via the corresponding partial pressures of the gas sources 1001, 1002, 1003.

For the physical simulation of inspiration, the gas module 100 comprises for example a vacuum pump 1004 together with valve and pressure sensor as inspiration unit 102 (see FIG. 1). Instead of a vacuum pump, it is alternatively or additionally possible to use other means that permit generation of an underpressure. In some embodiments, the gas sources 1001, 1002, 1003 and the vacuum pump 1004 act together as a combined inspiration and expiration unit. By suitable control of the gas sources, vacuum pump and valves, the breathing is thus physically simulated in the gas module 100.

Optionally, a valve 103 is additionally provided which is controlled in order to switch between inspiration and expiration. It is envisioned that the valve 103 is arranged and designed to permit stepless switching between the gas sources 1001, 1002, 1003 on the one hand and the vacuum pump 1004 on the other hand. For example, at the start of the simulated expiration, the valve 103 is switched such that gas from the gas sources is conveyed at least partially, in some embodiments mainly or exclusively, through the ducts to the port 105. By contrast, for the simulation of inspiration, the valve 103 is for example switched such that gas is conveyed from the port 105 in the direction of the vacuum pump 1004 or outlet 1014. The gas sources 1001, 1002, 1003 can for example be gas cylinders arranged in the gas module 100 or compressed gas ports and/or ports for external gas sources such as gas cylinders and/or compressed gas lines. Alternatively or in addition, provision can also be made that the vacuum pump 1004 is arranged in the gas module 100, but that an externally arranged vacuum pump (or source of underpressure) is connected to the gas module 100. If the valves of the gas sources 1001, 1002, 1003 are designed as “open-close” valves, then a further “open-close” valve can be arranged upstream of the vacuum pump 1004. For example, the valve 103 can then be omitted. The switching between inspiration and expiration then takes place for example via the switching of the respective valves.

In the embodiment shown in FIG. 2, the sensor arrangement 104 (see FIG. 1) comprises a pressure sensor 1005, a flow sensor 1006, a temperature sensor 1007 and a second pressure sensor 1008. Moreover, further sensors can be provided for the sensor arrangement 104, for example for detecting the gas composition and/or the gas humidity. The pressure sensor 1008 is arranged and designed to measure the air pressure of the ambient air. By means of the pressure sensor 1005, the flow sensor 1006 and the temperature sensor 1007, the pressure, flow and temperature of the gas of the entire simulated breathing (including any external influences) are detected which, when transferred to a living being, correspond to the values in the lungs and/or trachea. For example, if the gas is subjected to a pressure by the ventilator 900, the pressure sensor 1005 determines the gas pressure composed of the simulated breathing by the system 1 and of the pressure of the ventilator 900.

Besides the simulation of the active breathing, i.e. the at least partially autonomous breathing, of a living being, in some embodiments of the system provision is made that at least partially passive breathing is also able to be simulated, i.e. breathing specified for example by the connected ventilator 900. In some embodiments, the vacuum pump 1004 is suitably regulated for this purpose, and/or a bypass is provided via which the gas delivered by the ventilator 900 is conveyed past the vacuum pump 1004 to the outlet.

By way of the port 105 of the gas module 100, various appliances and/or means can be connected to the system 1 in a gas-conducting manner. For example, in FIG. 2, a ventilator 900 is connected to the system 1 via a connection 800. The ventilator 900 corresponds for example to a ventilator according to the prior art.

The system 1 further comprises a control module 200 for the mathematical simulation of the breathing or of the living being and for the control of the gas module 100. The control module 200 comprises a control unit 201 which, on the basis of control signals generated by the simulation unit 202, is designed and configured to control the gas module 100, in particular the gas sources 1001, 1002, 1003 and the valve 103 and also the vacuum pump 1004.

The sensor unit 203 is configured and designed to capture and optionally further process and/or condition the measurement values detected by the sensors 1005, 1006, 1007, 1008. The evaluation unit 204 is for example configured and designed to evaluate and optionally analyze the measurement values captured and optionally processed by the sensor unit 203. In some embodiments, provision is made that the simulation unit 202 uses the measurement values, captured by the sensor unit 203 and/or evaluated by the evaluation unit 204, as a basis for the mathematical simulation of the breathing and, if appropriate, adapts the control signals for the control unit 201. For example, it is possible to establish via the evaluation unit 204 and/or the simulation unit 202 that the physical simulation of the breathing does not coincide with the mathematical simulation. In this case, provision can be made that the simulation unit 202 and/or the control unit 201 suitably adapts the control of the gas module 100.

Moreover, the system 1 comprises an input unit 205. The input unit 205 can be used to input specifications, settings, values, data and/or information concerning the simulation of the breathing. Among other things, the analyses, evaluations and/or measurement values of the sensor unit 203 and of the evaluation unit 204 can be forwarded to the simulation unit 202 via the input unit 205. For example, an input module 300 via which inputs for the simulation can be made and data, values and/or information on the simulation of the breathing can be output and/or displayed is connected to the input unit 205. For example, the input unit 205 is for this purpose designed as a bidirectional interface which can receive and send data. For example, the input module 300 can be connected to the input unit 205 by a wired and/or wireless connection.

For example, the control module 200 comprises a storage unit 206. Measurement values, analyses and/or evaluations can be stored at least on an intermediate basis in the storage unit 206. In some embodiments of the system 1, provision is made that the mathematical simulation and also the measurement values detected by the sensor arrangement 104 are stored in the storage unit 206, for example for a later comparison between mathematical simulation and physical simulation. In some embodiments, a large number of simulation specifications and/or simulation sequences are stored in the storage unit 206 and can be accessed via the input module 300.

The input module 300 is in particular configured to input into the system 1 specifications and/or settings relating to the simulation of the breathing. The simulation unit 202 is for example configured and designed to mathematically simulate the breathing of the living being on the basis of the specifications and/or settings. The specifications and/or settings comprise by way of example, and not exclusively, pressure, flow, lung volume, gas composition, respiratory rate, tidal volume, type of living being, age, weight, diseases (in particular respiratory diseases), gas exchange, breathing problems. In some embodiments, provision is made that the input module 300 has an input mask via which settings relating to the simulation of the breathing are input, which are transmitted to the control module 200.

For the physical simulation of expiration, the system 1 shown by way of example in FIG. 2 is also configured to make available a gas mixture which simulates the gas composition of the air exhaled by a living being. For this purpose, a gas mixture is generated from the gas sources 1001, 1002, 1003 by suitable control of the valves. For example, the exchange of oxygen and CO₂ in the lungs of a living being can thus be simulated. In some embodiments, provision is made that the gas, which is conveyed into the system 1 during the simulated inspiration, is conveyed out of the system 1 via the outlet 1014, and a fresh gas or gas mixture is generated for the simulation of the expiration. In some embodiments, provision can also be made that the gas of inspiration is conveyed to the expiration unit 101 and gas is there admixed from the gas sources 1001, 1002, 1003, such that the composition corresponds to an exhaled gas.

For the simulation of the gas exchange in the lungs, provision can be made that the sensor arrangement 104 also comprises a sensor for determining the gas composition, in particular the oxygen concentration and/or CO₂ concentration. Through the analysis of the gas composition during the simulated inspiration, the simulation unit 202 calculates how the gas composition of the gas of the simulated expiration should be. In some embodiments, provision is also made that specifications relating to the simulated gas exchange can be made. For example, it is possible to stipulate that a low oxygen uptake in the lungs is intended to be simulated. Accordingly, for the simulated expiration, a gas mixture is generated which has a higher oxygen concentration than in the case of a normal gas exchange in the lungs.

Besides the gas sources shown in FIG. 2 for making available CO₂, oxygen and nitrogen, provision can be made that the expiration module 101 comprises further gas sources, for example one or more sources of anesthetic gas for the simulation of uptake of anesthetic gas in the lungs. Likewise, further gas sources can be provided, in each case corresponding to the gases which during inspiration are conveyed through the port 105 into the system 1 or the gas module 100 and whose uptake in the lungs and/or the airways is intended to be simulated. By way of the control module 200 and/or the input module 300, it is possible to set how much of the gas is taken up, wherein the corresponding gas composition is calculated for the simulated expiration, and the expiration module 101 is controlled accordingly.

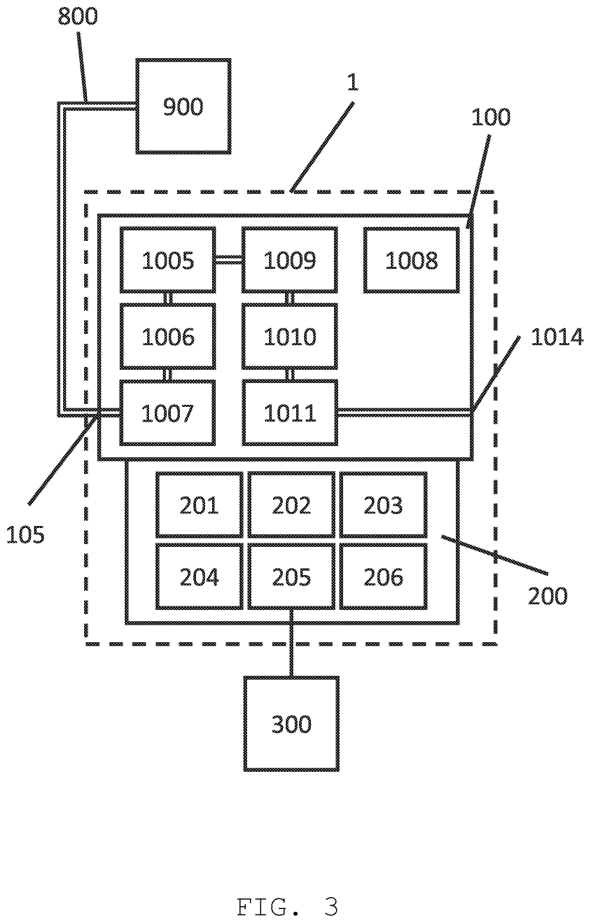

A further exemplary embodiment of the system 1 is shown schematically in FIG. 3. For the second simulation part, here the physical simulation of the breathing, two fans 1010, 1011 are arranged in the gas module 100. Alternatively or in addition to the two fans 1010, 1011, at least one bidirectional pump can also be used.

The fans 1010, 1011 have opposite output directions, such that both inspiration and expiration can be physically simulated. For example, the fan 1010 functions as expiration unit 101. Here, the output direction means in particular the pressure direction. For example, the fan 1010 builds up a pressure in the direction of the port 105 or ventilator 900. For example, the fan 1010 sucks gas through the outlet 1014 and feeds the gas through the gas module 100 to the port 105. The inspiration unit 102 is represented for example by the fan 1011, which is configured to convey gas counter to the output direction of the fan 1010. By means of the opposite output direction, i.e. suction of gas at the port 105, the inhalation of air int the trachea/lungs of a living being can be simulated, for example. The strength of the inspiration and of the expiration, for example in the form of pressure and/or flow, can be set among other things by the speed of the fans 1010, 1011. The tidal volume is correspondingly controllable over the duration of the delivery.

In addition to the fans 1010, 1011, a pneumatic resistance 1009 can be arranged in the gas module 100 for more precise and/or more extensive simulation of the breathing. For example, specific ratios of pressure and flow can be achieved via the pneumatic resistance 1009. In some embodiments, the pneumatic resistance 1009 serves primarily for better controllability of the flow. In some embodiments, the pneumatic resistance 1009 is controllable for this purpose. The pneumatic resistance 1009 can be adapted depending on the pressure/flow ratio to be obtained. For example, it is possible to physically simulate a large number of different breathing situations. In some embodiments, provision can be made to dispense with a pneumatic resistance 1009 or to use a fixed resistance, in which case a variable resistance is generated by the fans.

The fans 1010 and 1011 can in particular be controlled via the control unit 201. For example, for inspiration, only the fan 1011 is activated, the latter being arranged such that it sucks gas through the port 105 for the simulation of an active inspiration, i.e. the simulation of an autonomous inspiration of the living being. If the intention is to simulate an entirely passive inspiration by the living being, i.e. an inspiration with no autonomous drive, the fan 1011 can be at a standstill or be deactivated during the inspiration phase, and a bypass (not shown) can be opened by which the gas/gas mixture, e.g. respiratory gas, delivered from the ventilator 900 (or another ventilation source) is conveyed past the fans 1010, 1011 directly to the outlet 1014. During the simulated expiration, the fan 1010 remains deactivated.

The passive inspiration, i.e. ventilation by the ventilator 900, can be physically simulated even without a bypass. For example, one of the fans 1010, 1011 works with a pressure against the ventilation, for example in order to adjust a compliance. In passive ventilation, provision can be made that the gas delivered by the ventilator 900 can flow through the other and for example stationary fan.

For a simulated expiration, the fan 1010 is for example controlled via the control unit 201 such that a for example predefined expiration profile, at least as regards flow and pressure, is simulated. The fan 1011, which is used for the simulation of inspiration, remains deactivated during the expiration simulation. For the simulation of expiration, the fan 1010 is configured and designed to suck gas for example through the outlet 1014 and deliver it to the port 105. By varying the output rate of the fan 1010, optionally in combination with a pneumatic resistance 1009, an expiration profile can then be simulated. For example, at the start of the simulated expiration, a high flow is generated, which decreases in the course of the expiration phase.

The fans 1010, 1011 are for example configured and arranged such that gas can flow unimpeded through the fans counter to the delivery direction, without the fans being damaged, for example by constrained rotation of the conveying wheels counter to the envisioned direction. In some embodiments, provision is made that bypass lines are arranged in the gas module 100 such that gas is able to flow past the fans while the respective fan is deactivated.

While the control unit 201 is configured to control the fans 1010, 1011 and possibly the pneumatic resistance 109 such that the specifications of the mathematically simulated breathing are achieved, the resulting physically simulated breathing is tested via the sensor arrangement 104, for example via the pressure sensor 1005, the flow sensor 1006 and the temperature sensor 1007. The evaluation unit 204 is for example configured and designed to evaluate the measurement values of the sensors 1005, 1006, 1007 and to analyze them in order to ascertain whether the ventilation is simulated according to the specifications. In particular, the flow sensor 1006 is used to check whether the ventilation is (physically) simulated according to the specifications. The flow of the mathematical simulation serves as the specification, for example.

For example, the simulation unit 202, possibly in combination with the evaluation unit 204, is configured and designed to compare the physical simulation of the breathing with the mathematical simulation and to check for deviations. In some embodiments, the simulation unit 202 and/or the control unit 201 is configured to automatically carry out any corrections of the physical simulation. The simulation unit 202 is for example also configured and designed to incorporate into the mathematical simulation gas parameters, for example pressure and/or flow and/or temperature and/or gas composition, which are introduced from an external source, for example the ventilator 900, into the system 1. For example, in the mathematical simulation by the simulation unit 202, the pressure and/or flow generated by the ventilator 900 is included.

To generate a respiratory gas which for example physically simulates the consumption and production of respiratory gas components, it is possible in particular to provide an at least partial combination with the embodiment described with reference to FIG. 2. For example, the physical simulation of the inspiration and expiration and generally of the respiratory movement is realized by the fans 1010, 1011, while the gas mixture, for example as respiratory gas, is made available or adjusted by at least one gas source. In addition, provision can be made that a mixing region, for example a mixing chamber, is provided such that the respiratory gas delivered by the ventilator 900 can be effectively mixed with the gas made available from the at least one gas source. Correspondingly, provision can also be made that gas sensors are arranged in the region of the mixing chamber, for example at the inlet/outlet and/or in the mixing chamber itself, in order to monitor the gas composition. The additional gas can be fed in, for example, between the two fans 1010, 1011 and/or upstream and/or downstream of the two fans 1010, 1011. In particular, provision can be made that it is fed in between the fans and the port of the ventilator 900.

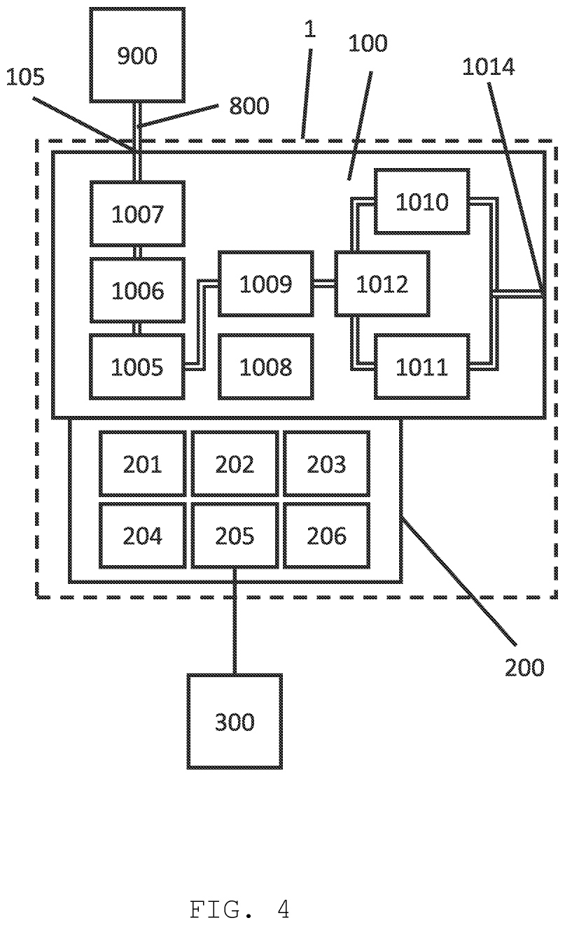

FIG. 4 shows an exemplary embodiment of the system 1 in conjunction with a ventilator 900 which is attached to the system 1 via a connection 800 to the port 105. Similarly to the embodiment described with reference to FIG. 3, the physical simulation of the breathing is also realized here via two fans 1010, 1011. In the embodiment shown in FIG. 4, the fans 1010, 1011 are for this purpose arranged parallel to each other. A valve 1012 is arranged in the gas module 100 such that it is possible to switch between the fans 1010, 1011.

For example, the expiration is simulated by the fan 1010. The system 1 is configured and designed such that the control unit 201 switches the valve 1012 so that a gas flow from the outlet 1014 to the port 105 via the fan 1010 is possible. For the simulation of the expiration, the fan 1010 conveys gas from the outlet 1014 to the port 105.

For the simulation of the inspiration, the valve 1012 is switched such that a gas flow from the port 105 via the fan 1011 to the outlet 1014 is possible. In the simulation of an at least partially active inspiration, such that at least the respiratory effort of the living being is simulated, the fan 1011 is correspondingly activated. The fan 1011 is configured and arranged such that gas is sucked from the port 105 and conveyed to the outlet 1014. By way of the sensor arrangement 104, in particular the flow sensor 1006, the quantity of gas can be determined which flows into the system 1 during the simulated inspiration. In accordance with a predefined, simulated lung volume, it is thus possible, for example, to control for how long and/or with which flow the inspiration is simulated.

In the simulation of an at least partially passive inspiration, in which the ventilator 900 largely determines the inspiration, the fan 1011 for example is deactivated. In some embodiments, provision is made that the fan 1011 generates at least a slight flow or pressure in order to simulate a respiratory effort. When the ventilator 900 switches to the ventilation, provision can be made that, by way of a bypass line in the gas module 100, the gas delivered by the ventilator is conveyed past the fan 1011 to the outlet 1014.

If a predefined simulated lung volume is reached, provision can be made that the valve 1012 is closed such that no further gas can be conveyed through the port 105 into the gas module 100. In some embodiments, provision can be made that, when the simulated lung volume is reached, the fan 1010 is also activated in order to simulate a counterpressure, which simulates a complete extension or filing of the lungs. Provision can also be made that the valve 1012 is switched when the mathematical simulation specifies that the pressure generated by the living being reaches zero, i.e. the living being has completed the inhalation or the exhalation and transitions to the next respiratory phase.

Alternatively or in addition, the pneumatic resistance 1009 can also be designed to be variable, such that the flow resistance is increased, which simulates an increasingly filled lung. In some embodiments, a fixed pneumatic resistance 1009 can also be provided, which at least serves to stabilize the flow regulation.

For the control of the gas module 100 and for the mathematical simulation of the breathing, the control module 200 is provided in the system 1. The simulation unit 202 establishes a mathematical simulation of the breathing, for example on the basis of specifications which are made for example by the input module 300, which is connected to the input unit 205 of the control module 200. In some embodiments, the measurement values detected by the sensor arrangement 104, comprising the sensors 1005, 1006, 1007, and captured and optionally evaluated by the sensor unit 203 and/or evaluation unit 204, can also be included by the simulation unit 202 in the mathematical simulation of the breathing. Provision is also made that the storage unit 206 is used to store simulation specifications and/or simulation sequences which can be called up via the input module and are optionally adaptable. For example, the simulation specifications and/or simulation sequences comprise information/data concerning the living being to be simulated. Provision can also be made that, in the simulation sequences, it is ascertained whether and/or which respiratory events are to be simulated, for example apnea and/or airway obstructions and/or changes in pressure/flow/frequency/volume of the breathing.

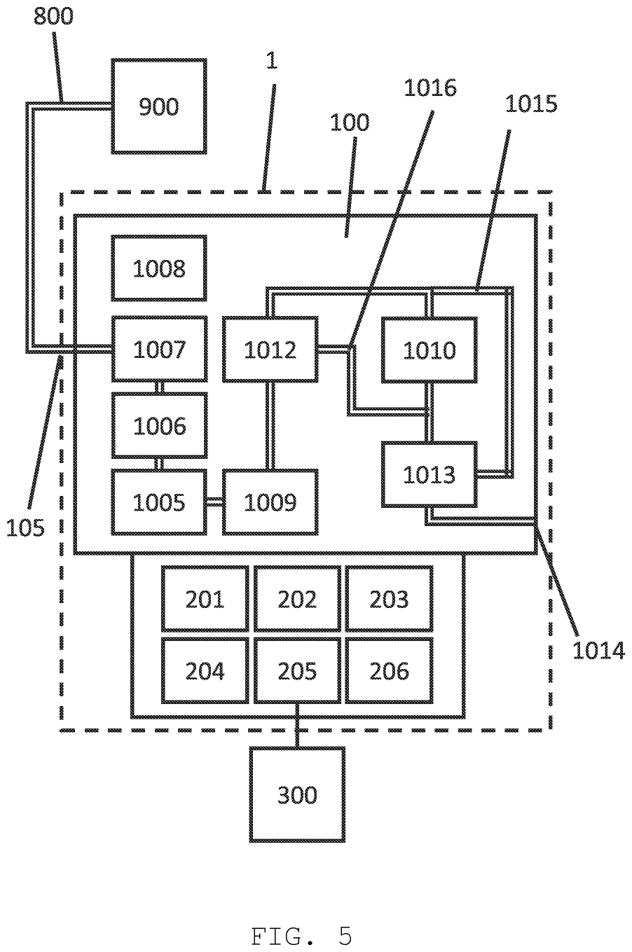

FIG. 5 shows a schematic representation of a further exemplary embodiment of the system 1 for simulating the breathing of a living being. The physical simulation of the breathing in the second simulation part, i.e. both the simulated inspiration and the simulated expiration, is simulated by the fan 101 in combination with the valves 1012 and 1013. The fan 1010, together with the valves 1012 and 1013, constitutes a combined expiration unit 101 and inspiration unit 102 (cf. FIG. 1).

Depending on the delivery direction or pressure direction of the fan 1010, it is decided, via the valves 1012, 1013, whether an inspiration or an expiration is simulated. For example, the valves 1012 and 1013 are switched when the pressure to be simulated reaches zero. The pressure to be simulated is the pressure which, in the mathematical simulation, corresponds to the pressure generated by the living being to be simulated. This largely corresponds to the natural breathing of a human/mammal which, upon exhalation, generates a positive pressure in the lungs, by which the gas is forced out of the lungs, and, upon inhalation, sucks gas into the lungs by means of an underpressure. It will be noted here that the pressure generated by the lungs or the living being is so described. For example, if a ventilator 900 which additionally generates a pressure is connected to the gas module, the pressure generated by the simulation is additionally subjected to the pressure of the ventilator 900. Thus, it may also happen that a positive pressure is measured constantly within the gas module. Through the simulation of the breathing, the pressure thus fluctuates around the pressure generated by the ventilator 900, depending on the compensation by the ventilator 900.

The valves 1012, 1013 are configured to convey the gas through the bypass lines 1015, 106. For example, the fan 1010 is configured and designed such that, in a first valve setting of the valves 1015, 1016, gas is sucked from the outlet 1014 and through the valve 1013 and is then conveyed through the valve 1012 to the port 105. The gas is not conveyed through the bypass lines 1015, 1016. The expiration of the living being is simulated by this delivery direction and gas routing.

In a second valve setting, the valves 1012, 1013 are set such that the gas is conveyed through the bypass lines 1015, 1016. In this way, the fan 1010 sucks gas from the port 105 via the valve 1012 and through the bypass line 1016 and conveys the gas via the bypass line 1015 through the valve 1013 to the outlet 1014. In this second valve setting, an at least partially active inspiration of the living being is accordingly simulated. By switching from the first valve setting to the second valve setting, the delivery direction of the gas through the gas module 100 can be reversed.

In some embodiments, a third valve setting is provided in which the valves 1012, 1013 are switched such that the gas is conveyed by valve 1012 through the bypass line 1015 past the fan 1010 and through the valve 1013 to the outlet 1014. For example, this third valve setting is set when an at least partially passive inspiration of a living being is to be simulated. In some embodiments, in this third valve setting the valve 1012 is switched such that the gas is conveyed into the bypass line 1016 and is conveyed through the valve 1013 directly to the outlet 1014. This switching affords the possibility that gas can be sucked at least slightly from the port 105 via the fan 1010, for example in order to simulate the effort made by the living being during inhalation. For example, it is possible to simulate a situation where the living being displays a respiratory effort, but the latter is not sufficient to permit complete inhalation, and external ventilation is needed, for example via the ventilator 900.

A specific pressure-to-flow ratio can be realized via the pneumatic resistance 1009, which is optionally variable. If the pneumatic resistance 1009 is designed to be variable, it is possible to simulate a large number of pressure-to-flow ratios, for example in order to simulate different diameters of the trachea and/or different breathing patterns or breathing problems. In some embodiments, the pneumatic resistance also or mainly serves for stabilizing the flow regulation.

The gas module 100 is controlled via the control module 200, in particular the control unit 201. The control signals are for example derived via the simulation unit 202 from the mathematical simulation of the breathing. The specifications concerning the breathing are input via the input module 300, for example. The specifications can relate, for example, to lung volume, flow, pressure, tidal volume, respiratory frequency, the simulation sequence, height, weight, diseases, etc., of the living being whose breathing is intended to be simulated. In some embodiments, inputs concerning the pressure and flow of the breathing to be simulated can at least be input. In some embodiments, the system 1 is designed to simulate not just the breathing of humans but also the breathing of other living beings, in particular mammals. For this purpose, the adjustable specifications would also comprise, for example, a choice of the respective living being.

The storage unit 206 can be used to store specifications, for example for living beings to be simulated and/or simulation sequences. These specifications can be adapted, for example. For example, it is possible to store simulation sequences which contain a large number of airway problems. For example the function of the attached ventilator 900 can thus be tested. In some embodiments, provision can be made that the simulation of the breathing can also be adapted during the simulation via the input module 300.

According to the specifications, in a first simulation part, a mathematical simulation of the breathing is effected by the simulation unit 202. Corresponding control signals are derived from the mathematical simulation of the breathing and transmitted to the control unit 201. On the basis of the control signals, the control unit 201 controls the gas module 100 in a second simulation part, for example in order to implement a physical simulation of the breathing.

A sensor arrangement 104 (cf. FIG. 4) is also arranged in the gas module 100 and comprises at least two pressure sensors 1005, 1008, a flow sensor 1006 and a temperature sensor 1007. The pressure sensor 1008 serves to measure the ambient pressure. Gas parameters such as flow, pressure, temperature are measured via the sensors 1005, 1006, 1007. The values measured here represent the gas parameters as occur in the lungs and the trachea of the living being whose breathing is simulated. The measurement values of the sensors 1005, 1006, 1007, 1008 are captured by the sensor unit 203 and optionally further processed. The evaluation unit 204 of the control module 200 is configured and designed to evaluate and/or analyze the measurement values captured by the sensor unit 203. In some embodiments, the control module 200 is configured and designed such that the control of the simulation is adapted on the basis of the analyses/evaluations of the evaluation unit 204. In some embodiments, the analyses/evaluations are forwarded via the input unit 205 to the simulation unit 202, such that a possible adaptation of the simulation takes place there and suitably adapted control signals are transmitted to the control unit 201.

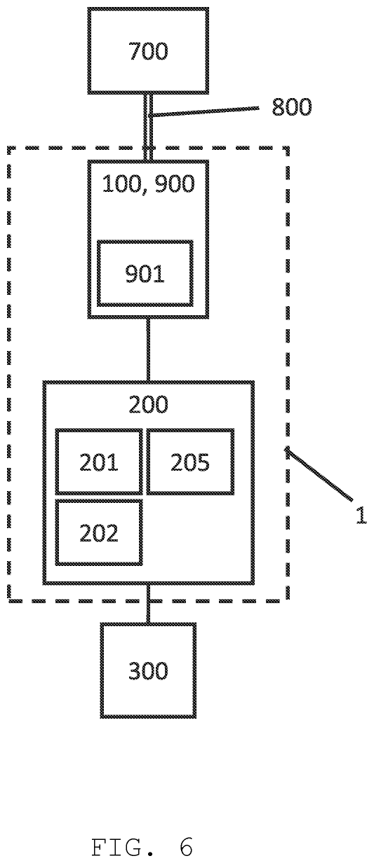

FIG. 6 shows an exemplary embodiment of the system 1, in which a ventilator 900 is used as gas module 100, and a real patient 700 is connected to the system 1 via the connection 800. The ventilator 900 can be a ventilator according to the prior art.

The system 1 is configured such that the ventilator 900 is controlled on the basis of the mathematical simulation of the breathing of the attached patient 700. Data and values relating to the breathing of the patient 700 are captured via sensors. In some embodiments, provision is made that the ventilator 900 has corresponding sensors and optionally also means for further processing and evaluation. The values relating to the breathing of the patient 700 are forwarded via the input unit 205 to the simulation unit 202. The simulation unit 202 is configured and designed, on the basis of the values of the patient 700, to mathematically simulate the patient 700 in a first simulation part. For example, the mathematical simulation also comprises the simulation of the lungs or lung values and/or also further vital values of the patient 700. In some embodiments, provision is made that the mathematical simulation is used to calculate or simulate values of the patient which, for example, are not accessible via the sensors.

For example, for the system 1 shown in FIG. 6, provision is made that the ventilator 900 is controlled at least partially on the basis of the mathematical simulation of the patient 700. For this purpose, the simulation unit 202 is configured to derive control signals from the mathematical simulation. The control signals are forwarded to the control unit 201, which is configured either to directly control the ventilator 900 or to forward these control signals to a ventilation control 901. In this second simulation part, the ventilation of the patient 700 is thus controlled on the basis of the mathematical simulation of the first simulation part.

To sum up, the present invention provides:
1. A system for simulating the breathing of a living being which comprises at least a gas module and a control module, the control module being configured and designed, in a first simulation part, to mathematically simulate a breathing of a living being, and, in a second simulation part, to control the gas module on the basis of the mathematical simulation from the first simulation part.
2. The system of item 1, wherein the control module comprises a simulation unit, which is configured and designed to mathematically simulate the breathing of a living being.
3. The system of at least one of the preceding items, wherein the control module is configured and designed to control the gas module such that in the second simulation part the mathematical simulation of the first simulation part is converted into a physical simulation of the breathing of a living being.
4. The system of at least one of the preceding items, wherein the gas module comprises at least one expiration unit and at least one inspiration unit, the expiration unit being configured to simulate the expiration of a living being, and the inspiration unit being configured to simulate the inspiration of a living being.
5. The system of at least one of the preceding items, wherein the simulation unit is designed to calculate and/or simulate the pressure which is generated by the simulated living being in the lungs.
6. The system of at least one of the preceding items, wherein the gas module is designed and configured to physically simulate the pressure which is generated by the simulated living being in the lungs.
7. The system of at least one of the preceding items, wherein the gas module is connectable via a port to a ventilator.
8. The system of at least one of the preceding items, wherein the expiration unit comprises at least one gas source and/or at least one fan.
9. The system of at least one of the preceding items, wherein a mathematically simulated respiratory flow is physically simulated by at least one fan, and a simulated gas composition is achieved by at least one gas source.
10. The system of at least one of the preceding items, wherein the inspiration unit is configured and designed to generate an underpressure.
11. The system of at least one of the preceding items, wherein the expiration unit comprises a plurality of gas sources, the expiration unit being configured and designed to make available, on the basis of the mathematical simulation, a gas mixture which corresponds to the gas composition of the exhaled air of a living being.
12. The system of at least one of the preceding items, wherein a fan is arranged in the gas module, the fan serving both as expiration unit and as inspiration unit by a switching of valves and bypass lines arranged in the gas module.
13. The system of at least one of the preceding items, wherein the system further comprises a sensor arrangement which is configured and designed to detect values of the breathing.
14. The system of item 13, wherein the control module is configured and designed to incorporate the values detected via the sensor arrangement into the mathematical simulation, the control module comprising an evaluation unit which is configured and designed to evaluate and/or analyze the values detected via the sensor arrangement.
15. The system of item 14, wherein the evaluation unit is configured and designed to analyze the values detected via the sensor arrangement in order to ascertain whether the mathematical simulation is correctly implemented by the gas module.
16. The system of at least one of the preceding items, wherein the system further comprises an input unit via which data, values and/or information are input, the data, values and/or information serving at least in part as specifications for the mathematical simulation.
17. The system of item 16, wherein the input unit is configured and designed to input values and/or data and/or information from an evaluation unit into a simulation unit.
18. The system of at least one of the preceding items, wherein the system further comprises a respiratory gas humidifier and/or a respiratory gas heater.
19. The system of at least one of the preceding items, wherein the control module is configured and designed to at least partially control a ventilator on the basis of the mathematical simulation, the ventilator being connected to a real person.
20. The system of at least one of the preceding items, wherein the system is combinable with patient simulators.
21. A method for simulating the breathing of a living being, wherein the breathing of the living being is simulated in a first simulation part by a mathematical simulation and, in a second simulation part, a gas module is controlled on the basis of the mathematical simulation.
22. The method of item 21, wherein the mathematical simulation is converted directly into commands, and the gas module is controlled on the basis of the commands.
23. The method of at least one of items 21 and 22, wherein, in one method step, measurement values relating to breathing are captured via sensors and are incorporated into the mathematical simulation.
24. The method of at least one of items 21 to 23, wherein the mathematical simulation is adapted and/or modified automatically on the basis of the captured measurement values.
25. The method of at least one of items 21 to 24, wherein measurement values relating to the breathing of a real person are used for the mathematical simulation.

### LIST OF REFERENCE SIGNS

1 system

100 gas module

101 expiration simulator

102 inspiration simulator

103 valve

104 sensor arrangement

105 port

200 control module

201 control unit

202 simulation unit

203 evaluation unit

204 sensor unit

205 input unit

206 storage unit

300 input module

700 patient

800 connection

900 ventilator

1001 CO₂ source

1002 O₂ source

1003 N₂ source

1004 vacuum pump

1005 pressure sensor

1006 flow sensor

1007 temperature sensor

1008 ambient pressure sensor

1009 pneumatic resistance

1010 fan

1011 fan

1012 two-way valve

1013 two-way valve

1014 outlet

1015 suction bypass

1016 suction bypass

9001 ventilation control

What is claimed is:

1. A system for simulating the breathing of a living being, wherein the system comprises at least a gas module and a control module, the control module being configured and designed, in a first simulation part, to mathematically simulate a breathing of a living being, and, in a second simulation part, to control the gas module on the basis of the mathematical simulation from the first simulation part.

2. The system of claim 1, wherein the control module comprises a simulation unit, which is configured and designed to mathematically simulate the breathing of a living being.

3. The system of claim 1, wherein the control module is configured and designed to control the gas module such that in the second simulation part the mathematical simulation of the first simulation part is converted into a physical simulation of the breathing of a living being.

4. The system of claim 1, wherein the gas module comprises at least one expiration unit and at least one inspiration unit, the expiration unit being configured to simulate an expiration of a living being, and the inspiration unit being configured to simulate an inspiration of a living being.

5. The system of claim 2, wherein the simulation unit is designed to calculate and/or simulate a pressure which is generated by the simulated living being in lungs.

6. The system of claim 1, wherein the gas module is designed and configured to physically simulate the pressure which is generated by the simulated living being in lungs.

7. The system of claim 1, wherein the gas module is connectable via a port to a ventilator.

8. The system of claim 4, wherein the expiration unit comprises at least one gas source and/or at least one fan.

9. The system of claim 1, wherein a mathematically simulated respiratory flow is physically simulated by at least one fan, and a simulated gas composition is achieved by at least one gas source.

10. The system of claim 4, wherein the inspiration unit is configured and designed to generate an underpressure.

11. The system of claim 4, wherein the expiration unit comprises a plurality of gas sources, the expiration unit being configured and designed to make available, on the basis of the mathematical simulation, a gas mixture which corresponds to a gas composition of exhaled air of a living being.

12. The system of claim 1, wherein a fan is arranged in the gas module, the fan serving both as expiration unit and as inspiration unit by a switching of valves and bypass lines arranged in the gas module.

13. The system of claim 1, wherein the system further comprises a sensor arrangement which is configured and designed to detect values of the breathing.

14. The system of claim 13, wherein the control module is configured and designed to incorporate values detected via the sensor arrangement into the mathematical simulation, the control module comprising an evaluation unit which is configured and designed to evaluate and/or analyze the values detected via the sensor arrangement.

15. The system of claim 14, wherein the evaluation unit is configured and designed to analyze the values detected via the sensor arrangement in order to ascertain whether the mathematical simulation is correctly implemented by the gas module.

16. The system of claim 1, wherein the system further comprises an input unit via which data, values and/or information are input, the data, values and/or information serving at least in part as specifications for the mathematical simulation.

17. The system of claim 16, wherein the input unit is configured and designed to input values and/or data and/or information from an evaluation unit into a simulation unit.

18. The system of claim 1, wherein the system further comprises a respiratory gas humidifier and/or a respiratory gas heater.

19. The system of claim 1, wherein the control module is configured and designed to at least partially control a ventilator on the basis of the mathematical simulation, the ventilator being connected to a real person.

20. A method for simulating the breathing of a living being, wherein the breathing of the living being is simulated in a first simulation part by a mathematical simulation and, in a second simulation part, a gas module is controlled on the basis of the mathematical simulation.
